(19) United States
(12) Patent Application Publication
(10) Pub. No.: US 2017/0246194 A1
(43) Pub. Date: Aug. 31, 2017

(54) Acetaminophen-Containing Analgesic Formulations With Reduced Hepatotoxicity

(71) Applicant: Purdue Pharma L.P., Stamford, CT (US)

(72) Inventor: Curtis WRIGHT, IV, Rockport, MA (US)

(73) Assignee: Purdue Pharma L.P., Stamford, CT (US)

(21) Appl. No.: 15/503,816

(22) PCT Filed: Aug. 14, 2015
(86) PCT No.: PCT/US2015/045379

§ 371 Date: Feb. 14, 2017

Related U.S. Application Data
(60) Provisional application No. 62/037,633, filed on Aug. 15, 2014.

(51) Int. Cl. A61K 31/616; A61K 31/485; A61K 31/192; A61K 31/4164; A61K 9/20; A61K 31/198; A61K 38/06; A61K 45/06; A61K 9/48; A61K 31/167; A61K 31/145

(52) U.S. Cl. CPC A61K 31/616; A61K 31/167; A61K 31/485; A61K 31/192; A61K 31/4164; A61K 31/145; A61K 31/198; A61K 38/063; A61K 45/06; A61K 9/48; A61K 9/20

(57) ABSTRACT

An analgesic composition is provided which is safe and effective to patients requiring pain modulation. The analgesic composition contains (a) acetaminophen and (b) at least three components chosen from (i) a bioavailable oral opioid; (ii) a non-steroidal anti-inflammatory drug; (iii) a sulfur-containing, N-acetyl-p-benzoquinone imine scavenging agent or prodrug thereof; or (iv) an agent which lowers acetaminophen toxicity. These analgesic compositions inhibit the formation or reduce the presence of NAPQI, the toxic acetaminophen by-product responsible for liver toxicity in patients. These analgesic compositions are also safely tolerated by patients administering more than the maximum dosage of acetaminophen.

### BACKGROUND

Acetaminophen (APAP) was introduced in the US in 1953 and in the United Kingdom in 1956. Acetaminophen is normally a safe analgesic. Taken in low doses, it is remarkably well tolerated, metabolized, and the metabolic products are excreted without incident. In overdose or in vulnerable individuals, however, the drug is toxic to the liver.

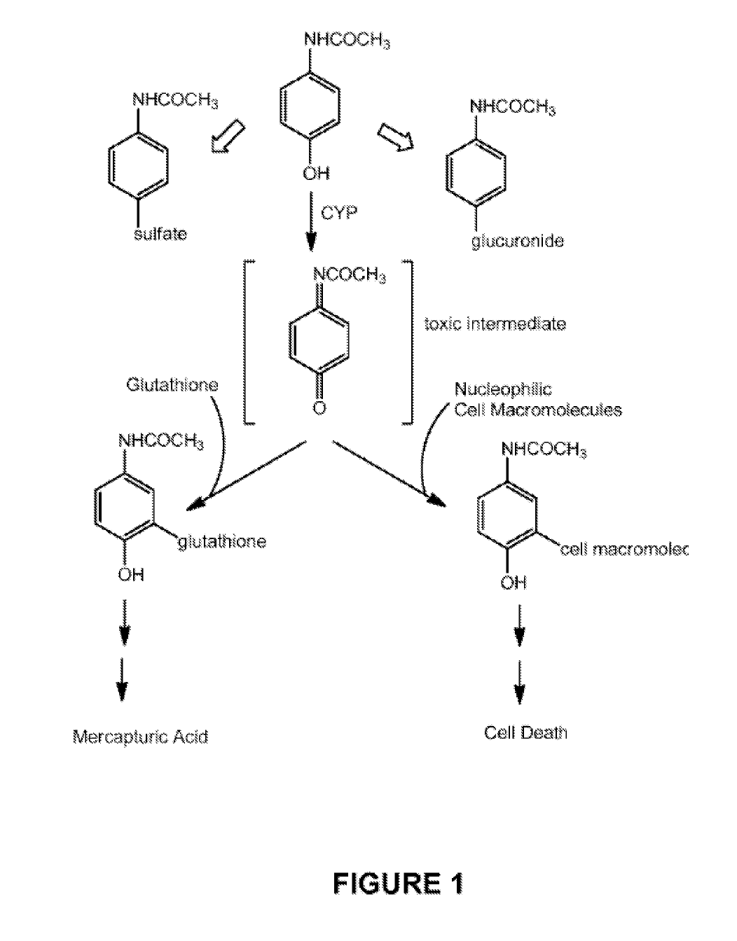

In the past 40 years, the metabolism of acetaminophen has been closely studied. Normally, 96% of the ingested dose of acetaminophen is metabolized to the non-toxic sulfate or the equally non-toxic glucuronide. A variable amount is metabolized by the cytochrome p-450 system (primarily CYP 2E1) to N-acetyl-p-benzoquinone imine (NAPQI). This compound is unstable, and will bind to essential cellular macromolecules, and lead to cell death, if not promptly scavenged by a glutathione scavenging process. If an excessive APAP dose is taken, or if the normal balance of detoxification is upset by up-regulation of the p450 system or by fasting, the glutathione-scavenging system is depleted and the toxic metabolite binds to essential cell structures leading to centrilobular necrosis of the liver. FIG. 1 depicts the metabolic pathway for acetaminophen in man and shows that there are multiple points of attack to prevent overdose injury.

Acetaminophen-opioid combinations are the most commonly prescribed analgesics in the United States. Hydrocodone-acetaminophen and oxycodone-acetaminophen combinations are used for short duration opioid analgesia, post-operative care, peak pain control for patients on non-steroidal anti-inflammatory drugs (NSAIDs), and general medical usage. They are acceptably safe drugs, but have a major safety concern that dominates their usage. Opioid-acetaminophen combinations are associated with drug induced hepatotoxicity in the United States. In some studies and populations, there are more combination analgesic liver toxicity cases than all other drug-induced hepatic injuries combined. Some studies have shown that 18-20% of patients prescribed these agents were prescribed a potentially toxic dose, that the post-treatment annual hospitalization rates for users runs 0.169% (169:100,000), and that there is an annual rate of hepatic or renal dysfunction of 2.3% which occurs among those regularly prescribed a toxic dose (>4 g/day). The population at risk is adults 40 years of age and over; they are four times more likely than a younger population to experience a combination analgesic overuse/overdose.

Acetaminophen, in overdose, alters its metabolism such that the toxic metabolite is formed. If not cleared from the liver, this toxin binds to liver cells, initiating an immune response resulting in destruction of the affected tissues.

Several agents have been studied in an effort to prevent or treat acetaminophen toxicity. Such agents include cimetidine, disulfiram, cysteamine, N-acetylcysteine, cysteine, glutathione, glutathione mono-ethyl ester, and methionine, and much has been discussed and published about the same. It is clear, however, that once the injury is established, after the overdose, adding another agent or otherwise trying to scavenge the NAPQI does not improve the outcome.

Warnings from a number of sources of acetaminophen-related acute liver failure have brought this problem before the authorities in Europe and the United States. For the past 30 years, the US FDA has been strengthening the warnings on OTC acetaminophen analgesics, adding specific language regarding alcohol use and asking manufacturers to limit combination analgesics to 325 mg of acetaminophen per dose. The FDA also recently added a recommendation to prescribers, and announced its intention to take action against any combination product that exceeded 325 mg of acetaminophen in content, as well as stating that one element of the long-term plan may be to take the combinations off the market, despite findings that treatment with opioids alone did not reduce the risk significantly.

Acetaminophen combinations containing narcotics are abused by taking “excessive” doses, but there is no commonly accepted number for the “abusable” or “excessive” dose of these medications. Most research studies maintain addicts on the equivalent of 120 mg of opioid (morphine equivalents) a day. Very few opioid addicts ever exceed 240 mg of opioid per day abusing prescription medications. This is often for reasons of cost, as a 240 mg a day habit would be $240 a day, $1680 a week. If an abuser were to try to reach 120 mg a day using a 5 mg opioid/325 mg acetaminophen product, the resulting 24 tablets a day would provide 7,800 mg of acetaminophen (about 2-2.5 times the currently recommended maximum daily dose of 3,000-3,250 mg). Since alcohol abuse is common in such a population, already making them more vulnerable to the toxic effects of acetaminophen, this is a dangerous and potentially liver-killing dose.

In the US, the epidemiology of acute liver failure due to acetaminophen is different from that in the UK. Severe acetaminophen toxicity in the UK is more likely to be related to intentional self-harm, and less likely to be related to overuse (>4 g/day of acetaminophen) or abuse of a prescribed opioid acetaminophen combination product.

To date, various strategies in the prior art have not provided a large enough protective effect against liver toxicity. What is needed in the art are acetaminophen-opioid combination analgesics that greatly reduce the risk and severity of acetaminophen hepatotoxicity, while maintaining the effectiveness of the analgesic and treating the patient's pain.

### SUMMARY OF THE INVENTION

In one aspect, an analgesic composition is provided and contains (a) acetaminophen and (b) at least three components chosen from (i) a bioavailable oral opioid; (ii) a non-steroidal anti-inflammatory drug; (iii) a sulfur-containing, N-acetyl-p-benzoquinone imine scavenging agent or prodrug thereof; or (iv) an agent which lowers acetaminophen toxicity. In one embodiment, the analgesic composition of the present invention comprises (a) acetaminophen and (b) three components chosen from (i), (ii), (iii), or (iv). In another embodiment, the analgesic composition of the present invention comprises (a) acetaminophen and (b) a component chosen from each of (i), (ii), (iii), and (iv).

In a further aspect, an analgesic composition is provided and contains (a) acetaminophen and (b)(i) oxycodone, hydrocodone, or codeine, (b)(ii) ibuprofen or aspirin, (b)(iii) cimetidine or disulfiram, and (b)(iv) methionine. In one embodiment of this aspect of the invention, (b)(i) is oxycodone. In another embodiment, (b)(i) is hydrocodone. In another embodiment, (b)(i) is codeine. In one embodiment, (b)(ii) is ibuprofen. In another embodiment, (b)(ii) is aspirin. In one embodiment, (b)(iii) is cimetidine. In another embodiment, (b)(iii) is disulfiram.

In another aspect, a method for inhibiting formation or reducing levels of N-acetyl-p-benzoquinone imine is provided and includes administering an analgesic composition described herein to a patient in need thereof.

In yet a further aspect, a method reducing toxicity of N-acetyl-p-benzoquinone imine is provided and includes administering an analgesic composition described herein to a patient in need thereof.

In still another aspect, a method for preventing or minimizing acetaminophen liver toxicity is provided and includes administering an analgesic composition described herein to a patient in need thereof.

In a further aspect, a method for preventing or treating pain is provided and includes administering an analgesic composition described herein to a patient in need thereof.

In yet another aspect, a method for treating pain or an ailment in a patient requiring treatment with acetaminophen is provided and includes administering at least one dose of an analgesic composition as described herein to a patient. In one embodiment, the hepatotoxic effects of acetaminophen administration are reduced with respect to administration of an equal amount of an analgesic composition comprising acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity. In another embodiment, the analgesic composition is administered to the patient without irreparable hepatic toxicity effects.

In still a further aspect, a method for treating pain or an aliment in a patient requiring treatment with acetaminophen is provided and includes administering an analgesic composition described herein to a patient in need thereof. In one embodiment, the risks of hepatotoxic effects of acetaminophen administration with self-administration of excessive doses are reduced with respect to administration of an equal amount of an analgesic composition containing acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity in the proper proportions. In another embodiment, the analgesic composition is administered to the patient so that excessive self-administration with multiples of the maximal recommended doses does not result in serious risk of irreparable hepatic toxicity effects. In another aspect, a method for decreasing acetaminophen induced hepatotoxicity in a patient is provided and includes administering an analgesic composition to the patient. In another aspect, the present disclosure provides a kit comprising: (A) an oral dosage unit comprising an analgesic composition as described above; and (B) instructions for administering said oral dosage unit.

In another aspect, the present disclosure provides an analgesic composition as described herein for use in a method for inhibiting formation or reducing levels of N-acetyl-p-benzoquinone imine in a patient.

In another aspect, the present disclosure provides an analgesic composition as described herein for use in a method for inhibiting formation or reducing toxicity of N-acetyl-p-benzoquinone imine in a patient.

In another aspect, the present disclosure provides an analgesic composition as described herein for use in a method for preventing or minimizing acetaminophen liver toxicity in a patient.

In another aspect, the present disclosure provides an analgesic composition as described herein for use in a method for preventing or treating pain in a patient.

In another aspect, the present disclosure provides an analgesic composition as described herein for use in a method for treating pain or an ailment in a patient requiring treatment with acetaminophen, wherein hepatotoxic effects of acetaminophen are reduced with respect to treatment with an equal amount of a composition comprising acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity.

In another aspect, the present disclosure provides an analgesic composition as described herein for use in a method for treating pain or an ailment in a patient requiring treatment with acetaminophen, wherein risks of hepatotoxic effects of acetaminophen administration with self-administration of excessive doses are reduced with respect to administration of an equal amount of a composition comprising acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity in the proper proportions; wherein excessive self-administration with multiples of the maximal recommended doses do not result in serious risk of irreparable hepatic toxicity effects.

In another aspect, the present disclosure provides an analgesic composition as described herein for use in a method for decreasing acetaminophen induced hepatotoxicity in a patient.

Other aspects and advantages of the invention will be readily apparent from the following detailed description of the invention. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE FIGURE

FIG. 1 provides the metabolic breakdown of acetaminophen after oral ingestion by a human.

### DETAILED DESCRIPTION OF THE INVENTION

Analgesic compositions, as described herein, reduce the risk of severe liver injury to all patients, including patients requiring pain prevention or alleviation under conditions of self-administration. Specifically, it is expected that the doses of the analgesic compositions described herein will result in less injury, fewer toxic intermediates, and lower levels of inflammatory injury to the liver.

The analgesic compositions discussed herein also make it possible for abusers to increase the amount of the addictive drug composition, i.e., an estimated three to six times the usual dose, without significant risk of liver toxicity. The analgesic compositions discussed herein permit maximizing of the amount of opioid relative to the non-opioid analgesic, while simultaneously minimizing of the amount of acetaminophen. In doing so, the compositions discussed herein block or minimize the conversion of acetaminophen to the toxic NAPQI metabolite and, if formed, immediately detoxify the formed NAPQI before it can injure cells. In one embodiment, the analgesic compositions discussed herein have an at least 150% increase in the toxic dose relative to acetaminophen administered alone. In another embodiment, the analgesic compositions discussed herein produce at least 200% increase in the toxic dose relative to single-entity acetaminophen. In one embodiment, surprisingly, the combination of a metabolic pathway blocker, a glutathione source, a reduced acetaminophen dose, and in certain instances an increased opioid ratio provides a clinically meaningful level of protection against liver toxicity.

Advantageously, the analgesic compositions discussed herein lack CYP 450-inducing or -stimulating additives or excipients. Specifically, stimulation of these enzyme systems acts to increase the toxicity of acetaminophen. In one embodiment, the CYP 450-inducing or -stimulating additives include, without limitation, caffeine, theophylline, or theobromine.

A. General Definitions

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of or “consisting essentially of language.

As used herein, the term “about” means a variability of 10% from the reference given, unless otherwise specified.

A “subject” or “patient” is a mammal, e.g., a human, mouse, rat, guinea pig, dog, cat, horse, cow, pig, or non-human primate, such as a monkey, chimpanzee, baboon or gorilla. Preferably, the mammal is a human.

As used herein, “disease”, “disorder” and “condition” are used interchangeably, to indicate an abnormal state in a subject.

The term “stable” as used herein refers to the analgesic compositions which degrade less than about 4%. In one embodiment, the analgesic compositions are stable at about 20° C./50% relative humidity to about 45° C./75% relative humidity. In another embodiment, the analgesic compositions degrade less than about 4% over a period of greater than 1 month at temperatures at or greater than about 25° C. and a relative humidity at or greater than about 60%. In a further embodiment, the analgesic compositions are stable over a period of about 2 months at temperatures of about 2 to about 8° C., optionally in the absence of light and moisture.

Unless defined otherwise in this specification, technical and scientific terms used herein have the same meaning as commonly understood by one of ordinary skill in the art and by reference to published texts, which provide one skilled in the art with a general guide to many of the terms used in the present application.

B. Analgesic Compositions

The analgesic compositions herein contain (a) acetaminophen and (b) at least three additional components chosen from (i) a bioavailable oral opioid, (ii) a non-steroidal anti-inflammatory drug, (iii) a sulfur-containing, N-acetyl-p-benzoquinone imine scavenging agent or prodrug thereof, or (iv) an agent which lowers acetaminophen toxicity.

In one embodiment, the analgesic composition contains acetaminophen, a bioavailable oral opioid, a non-steroidal anti-inflammatory drug, and a sulfur-containing, N-acetyl-p-benzoquinone imine scavenging agent or prodrug thereof. In another embodiment, the analgesic composition contains acetaminophen, a bioavailable oral opioid, a non-steroidal anti-inflammatory drug, and an agent which lowers acetaminophen toxicity. In a further embodiment, the analgesic composition contains acetaminophen, a bioavailable oral opioid, a non-steroidal anti-inflammatory drug, a sulfur-containing, N-acetyl-p-benzoquinone imine scavenging agent or prodrug thereof, and an agent which lowers acetaminophen toxicity. In still another embodiment, the analgesic composition contains acetaminophen, a bioavailable oral opioid, a sulfur-containing, N-acetyl-p-benzoquinone imine scavenging agent or prodrug thereof, and an agent which lowers acetaminophen toxicity. In other embodiments, combinations containing acetaminophen and components (i)-(iv) are envisioned even though not specifically recited.

As discussed above, it is advantageous that the analgesic compositions lack a CYP450-inducing or CYP450-stimulating additive, typically caffeine, as stimulating these enzyme systems increases the production of toxic metabolites of acetaminophen. In one embodiment, the CYP450-inducing or CYP450-stimulating additive is caffeine, theophylline, or theobromine. In a further embodiment, the analgesic compositions discussed herein lack caffeine.

(i) The Analgesic Component

As a first component, the analgesic compositions discussed herein contain acetaminophen as the analgesic. The term “analgesic” as used herein refers to a pharmaceutical compound which prevents or relieves pain in a subject in need thereof. The term “analgesic” as defined herein excludes agents that act as non-steroidal anti-inflammatory agents (NSAIDs). The term “acetaminophen” as used herein refers to an aniline analgesic having the chemical name of N-acetyl-p-aminophenol. The term acetaminophen is also synonymous with paracetamol. Acetaminophen is available in the art and in over-the-counter forms under brand names such as Tylenol®, Panadol®, and Mapap®.

The analgesic composition contains sufficient amounts of acetaminophen which is required to act as an analgesic and, thereby, contribute to treating the ailment suffered by the patient. In one embodiment, the amount of acetaminophen in the analgesic compositions discussed herein is less than the amount of acetaminophen in the usual formulations available in the art. By doing so, there is less of a risk in overdosing, accidentally or otherwise, and damaging the liver. It is an advantage of the present analgesic compositions that the amount of acetaminophen is about 20 to about 25%, or integers, fractions, or ranges there between, less than the FDA recommended maximum amount (i.e., 325 mg). In one embodiment, the amount of acetaminophen is about 20, 21, 22, 23, 24, or 25% less than the FDA recommended maximum amount.

The normal dose of combination analgesics is about 5 mg of a bioavailable oral opioid such as hydrocodone or oxycodone, along with about 325 mg (per dose) of acetaminophen. Taken as directed, the usual daily dose is 1 to 2 tablets every about 4 to about 6 hours while awake. In one embodiment, this gives a daily dose of about 20 to about 60 mg of opioid and about 1,300 to about 3,900 mg of acetaminophen. In another embodiment, this includes daily administering about 4 to about 12 tablets of the combination analgesic.

In one embodiment, the analgesic composition contains from about 20 to about 80% wt/wt, or integers, fractions, or ranges there between, of acetaminophen. In a further embodiment, the analgesic composition contains about 20, 25, 30, 35, 40, 45, 50, 55, 60, 65, 70, 75, or 80% wt/wt of acetaminophen. In another embodiment, the analgesic composition contains from about 35 to about 65% wt/wt of acetaminophen. In another embodiment, the analgesic composition contains from about 40 to about 45% wt/wt of acetaminophen. In a further embodiment, the analgesic composition contains from about 150 to about 500 mg, or fractions, integers, or ranges there between, of acetaminophen. In still another embodiment, the analgesic composition contains about 150, 200, 250, 300, 350, 400, 450, or 500 mg of acetaminophen. In yet another embodiment, the analgesic composition contains about 250 mg of acetaminophen. In still another embodiment, the analgesic composition contains about 300 mg of acetaminophen. In a further embodiment, the analgesic composition contains about 325 mg of acetaminophen. In still another embodiment, the analgesic composition contains about 400 mg of acetaminophen. In a further embodiment, the analgesic composition contains about 500 mg of acetaminophen. In still another embodiment, the analgesic composition contains about 650 mg of acetaminophen.

Additional analgesics may be included in the analgesic compositions discussed herein and may be selected by one skilled in the art. In one embodiment, the additional analgesic may be chosen from, without limitation, COX-2 inhibitors (celecoxib, rofecoxib), tricyclic antidepressants (amitriptyline and duloxetine), atypical analgesics (Nefopam, orphenadrine, gabapentin, pregabalin, cyclobenzaprine, scopolamine), carbamazepine, anticonvulsants, psychotropic agents (tetrahydrocannabinol), flupirtine, hydroxyzine, promethazine, carisoprodol, tripelennamine, methadone, ketobemidone, and piritramide.

(ii) Opioid Component

One optional component of the analgesic composition discussed herein is an opioid (i.e., a bioavailable oral opioid). One of skill in the art may readily select an opioid for inclusion in the analgesic compositions and may be chosen from an opioid which is taken orally. Examples of oral opioids include, without limitation, oxycodone, hydrocodone, codeine, dihydrocodeine, hydromorphone, morphine, and propoxyphene. In one embodiment, the opioid is oxycodone, hydrocodone, or codeine. In one embodiment, the opioid is oxycodone. In another embodiment, the opioid is hydrocodone. In another embodiment, the opioid is codeine.

In an effort to reduce the toxic effects resulting from the acetaminophen component, the amount of opioid in the analgesic composition is high related to the amount of acetaminophen. In one embodiment, the weight ratio of the opioid to acetaminophen is about 1:10 or greater. In another embodiment, the weight ratio of the opioid to acetaminophen is about 1:25 or greater. In another embodiment, the weight ratio of the hydrocodone or oxycodone to acetaminophen is about 5:325 or greater. In a further embodiment, the weight ratio of codeine to acetaminophen is about 30:325 or greater.

Thus, the analgesic composition contains from about 0.5 to about 16% wt/wt, or fractions, integers, or ranges there between, of the opioid. In one embodiment, the analgesic compositions contains about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, or 16% wt/wt of the opioid. In a further embodiment, the analgesic composition contains from about 0.5 to about 3% wt/wt, or fractions, integers, or ranges there between, of oxycodone or hydrocodone. In another embodiment, the analgesic composition contains from about 1 to about 3% wt/wt of oxycodone or hydrocodone. In still a further embodiment, the analgesic composition contains about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0% wt/wt of oxycodone or hydrocodone. In another embodiment, the analgesic composition contains from about 2.5 to about 60 mg, or integers, fractions, or ranges there between, of the opioid. In still another embodiment, the analgesic composition contains about 2.5, 5, 7.5, 10, 15, 20, 22.5, 25, 30, 35, 37.5, 40, 45, 50, 52.5, 55, or 60 mg of the opioid. In one embodiment, the opioid is oxycodone or hydrocodone. In yet a further embodiment, the analgesic composition contains about 5 mg of the opioid. In another embodiment, the analgesic composition contains about 7.5 mg of the opioid. In a further embodiment, the analgesic composition contains about 10 mg of the opioid. In another embodiment, the analgesic composition contains about 10 mg of oxycodone or hydrocodone.

(iii) Non-Steroidal Anti-Inflammatory Drug Component

A second optional component of the analgesic composition is a non-steroidal anti-inflammatory drug (NSAID). The term “NSAID” as used herein refers to a pharmaceutical drug which provides analgesic and anti-inflammatory effects on a patient in need thereof. It is expected that the use of the NSAID in the analgesic composition permits the amount of acetaminophen in the analgesic composition to be reduced. In one embodiment, the NSAID acts as a co-analgesic and anti-inflammatory agent when given with acetaminophen alone or in mixtures of acetaminophen and a second NSAID analgesic. In one embodiment, the inclusion of the NSAID in the analgesic composition permits the amount of acetaminophen in the composition to be at least halved.

The analgesic composition, thereby, contains from about 10 to about 65% wt/wt, or integers, fractions, or ranges there between, of the NSAID. In one embodiment, the analgesic composition contains about 10, 15, 20, 25, 30, 35, 40, 45, 50, 55, 60, or 65% wt/wt of the NSAID. In another embodiment, the analgesic composition contains from about 50 to about 500 mg, or integers, fractions, or ranges there between, of the NSAID. In yet another embodiment, the analgesic compositions contains about 50, 100, 150, 200, 250, 300, 350, 400, 450, or 500 mg of the NSAID. In a further embodiment, the analgesic composition contains about 325 mg of the NSAID. In still another embodiment, the analgesic composition contains about 400 mg of the NSAID. In a further embodiment, the analgesic composition contains about 500 mg of the NSAID. In still another embodiment, the analgesic composition contains about 650 mg of the NSAID. In one embodiment, the amount of NSAID described above is that for aspirin, naproxen or ibuprofen. In another embodiment, the analgesic composition contains aspirin, for example, in an amount of from about 30 to about 45% wt/wt. In a further embodiment, the analgesic composition contains about 250 mg of aspirin. In yet another embodiment, the analgesic composition contains naproxen, for example, in an amount of from about 15 to about 30% wt/wt. In still a further embodiment, the analgesic composition contains about 110 mg of naproxen. In another embodiment, the analgesic composition contains ibuprofen, for example, in an amount of from about 30 to about 40% wt/wt. In yet a further embodiment, the analgesic composition contains about 200 mg of ibuprofen.

One or more NSAIDs may be included in the analgesic composition. One of skill in the art would be able to select a suitable NSAID for inclusion in the analgesic compositions discussed herein. Examples of suitable NSAIDs include, without limitation, salicylates such as aspirin (ASA), diflunisal, salsalate, or choline magnesium trisalicylate; propionic acid derivatives such as ibuprofen, dexibuprofen, naproxen, fenoprofen, ketoprofen, dexketoprofen, flurbiprofen, oxaprozin, or loxoprofen; acetic acid derivatives such as indomethacin, tolmetin, sulindac, etodolac, ketorolac, diclofenac, aceclofenac, or nabumetone; enolic acid derivatives such as piroxicam, meloxicam, tenoxicam, droxicam, lornoxicam, or isoxicam; anthranilic acid derivatives such as mefenamic acid, meclofenamic acid, flufenamic acid, or tolfenamic acid; selective COX-2 inhibitors such as celecoxib, rofecoxib, valdecoxib, prarecoxib, lumiracoxic, etoricoxib, or firocoxib; sulfonanilides such as nimesluide; licofelone; H-harpagide in Figwork or Devil's Claw; and lysine clonixinate. In one embodiment, the NSAID is chosen from aspirin, naproxen or ibuprofen.

(iv) Agent That Lowers Acetaminophen Toxicity Component

Another optional component of the analgesic composition described herein is an agent that lowers acetaminophen toxicity. Specifically, the agent that lowers acetaminophen toxicity works by reducing conversion of acetaminophen to its toxic metabolite, i.e., NAPQI.

One of skill in the art would readily be able to select an agent that lowers acetaminophen toxicity. In one embodiment, the agent that lowers acetaminophen toxicity is chosen from cimetidine or disulfiram. The analgesic composition contains from about 2 to about 25% wt/wt, or fractions, integers or ranges there between, of the agent that lowers acetaminophen toxicity. In one embodiment, the analgesic compositions contains about 2, 3, 4, 5, 6, 7, 8, 9, 10, 11, 12, 13, 14, 15, 16, 17, 18, 19, 20, 21, 22, 23, 24, or 25% of the agent that lowers acetaminophen toxicity. In a further embodiment, the analgesic composition contains from about 2 to about 20% wt/wt of the agent that lowers acetaminophen toxicity. In another embodiment, the analgesic composition contains from about 2 to about 8% wt/wt of the agent that lowers acetaminophen toxicity. In a further embodiment, the analgesic composition contains from about 20 to about 75 mg, or integers, fractions, or ranges there between, of the agent that lowers acetaminophen toxicity. In still a further embodiment, the analgesic compositions contains about 20, 25, 30, 35, 40, 45, 50, 55, 60, 65, 70, or 75 mg of the agent that lowers acetaminophen toxicity. In yet another embodiment, the analgesic composition contains about 25 mg of the agent that lowers acetaminophen toxicity. In a further embodiment, the analgesic composition contains about 35 mg of the agent that lowers acetaminophen toxicity. In still a further embodiment, the analgesic composition contains about 25 mg of the agent that lowers acetaminophen toxicity. In another embodiment, the analgesic composition contains about 50 mg of the agent that lowers acetaminophen toxicity. In one embodiment, the agent that lowers acetaminophen toxicity is cimetidine. In another embodiment, the agent that lowers acetaminophen toxicity is disulfiram.

(v) Sulfur-Containing Agent Component

A further optional component of the analgesic composition is a sulfur-containing agent. In one embodiment, the sulfur-containing agent assists in scavenging any undesired NAPQI generated in the patient. One of skill in the art would readily be able to select a suitable sulfur-containing agent for use as described herein. In one embodiment, the sulfur-containing agent is chosen from, without limitation, methionine, glutathione, n-acetyl-cysteine, or L-cysteine. In one embodiment, the sulfur containing agent is methionine. In another embodiment, the sulfur containing agent is glutathione. In another embodiment, the sulfur containing agent is n-acetyl-cysteine. In another embodiment, the sulfur containing agent is L-cysteine.

The analgesic composition contains from about 3 to about 35% wt/wt, or integers, fractions, or ranges there between, of the sulfur-containing agent. In one embodiment, the analgesic composition contains about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35% wt/wt of the sulfur-containing agent. In another embodiment, the analgesic composition contains from about 6 to about 15% wt/wt of methionine. In a further embodiment, the analgesic composition contains from about 6 to about 35% wt/wt of glutathione. In a further embodiment, the analgesic composition contains from about 6 to about 30% wt/wt of glutathione. In another embodiment, the analgesic composition contains about 35% wt/wt of n-acetyl-cysteine. In another embodiment, the analgesic composition contains from about 6 to about 35% wt/wt of n-acetyl-cysteine. In another embodiment, the analgesic composition contains from about 6 to about 30% wt/wt of n-acetyl-cysteine. In still another embodiment, the analgesic composition contains from about 6 to about 35% wt/wt of L-cysteine. In another embodiment, the analgesic composition contains from about 6 to about 30% wt/wt of L-cysteine. In yet a further embodiment, the analgesic composition contains from about 25 to about 125 mg, or integers, fractions, or ranges there between of the sulfur-containing agent. In another embodiment, the analgesic composition contains about 25, 30, 35, 40, 45, 50, 55, 60, 65, 70, 75, 80, 85, 90, 95, 100, 105, 110, 115, 120, or 125 mg of the sulfur-containing agent. In still another embodiment, the analgesic composition contains about 50 mg of the sulfur-containing agent. In a further embodiment, the analgesic composition contains about 75 mg of the sulfur-containing agent. In yet another embodiment, the analgesic compositions contains about 100 mg of the sulfur-containing agent. In another embodiment, the analgesic composition contains from about 50 to about 100 mg of methionine. In another embodiment, the analgesic composition contains about 50 mg of methionine. In another embodiment, the analgesic composition contains 75 mg of methionine. In another embodiment, the analgesic composition contains about 100 mg of methionine. In a further embodiment, the analgesic composition contains about 50 to about 150 mg of glutathione. In yet another embodiment, the analgesic composition contains about 50 to about 150 mg of n-acetyl-cysteine. In yet a further embodiment, the analgesic composition contains about 50 to about 150 mg of L-cysteine.

(vi) Additional Components of the Analgesic Composition

Additional components may be included in the components of the analgesic compositions described herein. In one embodiment, the additional components perform one or more active roles when administered to a patient. Such additional components include one or more, without limitation, antihistamines, stimulants, H1 blockers, H2 blockers, anti-inflammatories, proton-pump inhibitors, laxatives, 5-HT3-blockers, anxiolytics, glutathione production promoters, antiemetics, odor reducers, or muscle relaxants. One of skill in the art would be able to select suitable options for these active additional components. In one embodiment, the additional component is chosen from aluminum hydroxide, brompheniramine, butalbital, chlorpheniramine, clemastine, dexbrompheniramine, dextromethorphan, dichloralphenazone, diphenhydramine, doxylamine, guaifenesin, isometheptene mucate, magnesium hydroxide, magnesium salicylate, pamabrom, pentazocine, pheniramine, phenylephrine, phenylpropanolamine, phenyltoloxamine, propoxyphene, pseudoephedrine, pyrilamine, pyridoxine, salicylamide, tramadol, or tripolidine. In another embodiment, the glutathione production promoter is N-acetyl-cysteine.

In another embodiment, the additional components do not interfere with the function of the required components. The analgesic compositions can thereby further include, without limitation, one or more pharmaceutically acceptable excipients, such as adjuvants, diluents, binders, lubricants, glidants, surfactants, wetting agents, granulating agents, disintegrating agents, fragrances, emollients, pH adjustors, fillers, flavoring agents, coloring agents, preservatives, coating reagents, stability enhancing agents, such as antioxidants, and combinations thereof, among others. See, the pharmaceutical excipients discussed in Remington's Pharmaceutical Sciences, Handbook of Pharmaceutical Excipients, 7^{th} Ed., Rowe et al., Eds., 2012. In one embodiment, the antioxidant is butylated hydroxyl toluene, vitamin E, or a combination thereof. In one embodiment, the pharmaceutically acceptable excipient is chosen from a fragrance, a coloring agent or a stability enhancing agent.

The additional components can therefore include, without limitation, one or more of vitamin E, ascorbic acid, benzoic acid, butylated hydroxytoluene (BHT), butylated hydroxyanisole (BHA), calcium carbonate, calcium phosphate, calcium citrate, carnauba wax, casein, cetostearyl alcohol, cetyl alcohol or ester wax, cysteine, cellulose, cholesterol, carboxymethylcellulose calcium, carboxymethylcellulose sodium, colloidal silicone dioxide, croscarmellose sodium, crospovidone (polyplasdone), dextrate, dextrin, gelatin, glyceryl monooleate, gum arabic and acacia, hydroxymethylcellulose, hydroxypropylcellulose, hydroxyethyl cellulose, hydroxypropylmethylcellulose phthalate, hypromellose, isopropyl laurate, lecithin (phosphatide), magnesium stearate, mannitol, methylcellulose, 5-methyl-(6s)-tetrahydrofolate, microcrystalline cellulose, monostearate, myristyl myristate, noncrystalline cellulose, oleic acid, oils such as mineral oil, mink oil, olive oil castor oil derivative or stearate, oleyl alcohol, palmitic acid, palmitostearate, pectin, petroleum jelly, polyethylene glycols, polyoxyethylene alkyl ethers, polypropylpyrrolidone, polyvinylpyrrolidone (PVP), polysorbates such as polysorbate 20, povidone, powdered cellulose, propylene glycol, polyvinyl alcohol, silicic acid, simethicone, sodium bicarbonate, sodium lauryl sulfate (SLS), sodium metabisulfite, sodium starch glycolate, sodium stearyl furamate, sorbitan esters such as sorbitan monolaurate, poloxamer, edetic acid, malic acid, fumaric acid, citric acid, ascorbic acid, fumaric acid, or malic acid, starch, modified starch, pregelatinized starch, stearyl alcohol, shellac, stearic acid, sugar such as sucrose, sucralose, kaolin, dextrose, and lactose such as anhydrous lactose or lactose monohydrate, talc, tragacanth, titanium dioxide, triacetin, zinc salts such as zinc stearate, or combinations thereof.

In another embodiment, the analgesic compositions described herein may be combined with a probiotic bacterial strain for use in the kits, delivery devices, and methods described herein. Probiotic bacterial strains include, without limitation, Acidophilus lactobacillus, reuteri lactobacillus, casei lactobacillus, bifidobacterium lactus, or bifidobacterium bifidum.

These additional components, active or inactive, may be coarse milled and/or micronized, as described below prior to formulation. In one embodiment, each additional component is milled and/or micronized. In another embodiment, all of the additional components are milled and/or micronized together.

The amounts of the additional components in the analgesic compositions will be determined by the amounts of the active agents in the same. In one embodiment, these additional components are present in the analgesic compositions in an amount sufficient to total a 100% wt/wt of the final composition. One of skill in the art would readily be able to determine the amounts of these additional components in the analgesic compositions.

(vii) Analgesic Composition Embodiments

In one embodiment, an analgesic composition is provided and includes (a) acetaminophen and (b) at least three components chosen from (i) a bioavailable oral opioid; (ii) a non-steroidal anti-inflammatory drug; (iii) a sulfur-containing, N-acetyl-p-benzoquinone imine scavenging agent or prodrug thereof; or (iv) an agent which lowers acetaminophen toxicity.

In another embodiment, an analgesic composition is provided and includes (a) acetaminophen and (b)(i) a bioavailable oral opioid; (b)(ii) a non-steroidal anti-inflammatory drug; (b)(iii) a sulfur-containing, N-acetyl-p-benzoquinone imine scavenging agent or prodrug thereof; and (b)(iv) an agent which lowers acetaminophen toxicity.

In a further embodiment, an analgesic composition is provided and contains (a) acetaminophen and (b)(i) oxycodone, hydrocodone, or codeine, (b)(ii) aspirin, naproxen, or ibuprofen, (b)(iii) cimetidine or disulfiram, and (b)(iv) methionine.

In yet another embodiment, an analgesic composition is provided and contains (a) acetaminophen and (b)(i) oxycodone, hydrocodone, or codeine, (b)(ii) aspirin, (b)(iii) cimetidine or disulfiram, and (b)(iv) methionine.

In another embodiment, an analgesic composition is provided and contains (a) acetaminophen and (b)(i) oxycodone, (b)(ii) aspirin, (b)(iii) cimetidine, and (b)(iv) methionine.

In another embodiment, an analgesic composition is provided and contains (a) acetaminophen and (b)(i) hydrocodone, (b)(ii) aspirin, (b)(iii) cimetidine, and (b)(iv) methionine.

In another embodiment, an analgesic composition is provided and contains (a) from about 250 mg to about 650 mg of acetaminophen; and (b)(i) from about 5 to about 10 mg of oxycodone or hydrocodone; (b)(ii) from about 250 mg to about 650 mg of aspirin; (b)(iii) from about 0 mg to about 50 mg of cimetidine; and (b)(iv) from about 0 mg to about 100 mg of methionine, provided that at least one of cimetidine and methionine is present in the analgesic composition.

In another embodiment, an analgesic composition is provided and contains (a) from about 250 mg to about 650 mg of acetaminophen; (b)(i) from about 5 to about 10 mg of oxycodone or hydrocodone; (b)(ii) from about 250 mg to about 650 mg of aspirin; (b)(iii) from about 25 mg to about 50 mg of cimetidine; and (b)(iv) from about 50 mg to about 100 mg of methionine.

In another embodiment, an analgesic composition is provided and contains (a) from about 250 mg to about 650 mg of acetaminophen; (b)(i) from about 5 to about 10 mg of oxycodone or hydrocodone; (b)(ii) from about 250 mg to about 650 mg of aspirin; (b)(iii) from about 25 mg to about 50 mg of cimetidine; and (b)(iv) from about 50 mg to about 100 mg of methionine.

In another embodiment, an analgesic composition is provided and contains (a) about 250 mg of acetaminophen; (b)(i) from about 5 to about 10 mg of oxycodone or hydrocodone; (b)(ii) about 250 mg aspirin; (b)(iii) from about 25 mg to about 50 mg of cimetidine; and (b)(iv) from about 50 mg to about 100 mg of methionine.

In still a further aspect, an analgesic composition is provided and contains (a) about 250 mg of acetaminophen; (b)(i) about 10 mg of oxycodone or hydrocodone; (b)(ii) about 250 mg of aspirin; (b)(iii) about 50 mg of cimetidine or disulfiram; and (b)(iv) about 100 mg of methionine.

In another embodiment, an analgesic is provided and contains (a) about 250 mg of acetaminophen; (b)(i) about 30 mg of codeine; (b)(ii) about 250 mg of aspirin; (b)(iii) about 25 mg of cimetidine or disulfiram; and (b)(iv) about 50 mg of methionine.

In yet a further aspect, an analgesic composition is provided and contains (a) about 250 mg of acetaminophen; (b)(i) about 10 mg of oxycodone, hydrocodone, or codeine; (b)(ii) about 200 mg of ibuprofen; (b)(iii) about 50 mg of cimetidine or disulfiram; and (b)(iv) about 100 mg of methionine.

In another embodiment, an analgesic composition is provided and contains (a) about 250 mg of acetaminophen; (b)(i) about 10 mg of oxycodone, hydrocodone, or codeine; (b)(ii) about 200 mg of ibuprofen; (b)(iii) about 25 mg of cimetidine or disulfiram; and (b)(iv) about 50 mg of methionine.

(viii) Methods for Preparing the Analgesic Compositions

The analgesic compositions described herein may be prepared using a number of methods known to those skilled in the art. See, e.g., Lieberman, “Pharmaceutical Dosage Forms: Tablets”, Vol. 3, 1990, which are herein incorporated by reference. In one embodiment, the analgesic compositions described herein may be prepared by either direct compression or dry mixing/granulating or wet mixing granulating. The analgesic compositions are generally based upon the total weight of the unit dose with the other components of the analgesic composition to form a dry granulation. In another embodiment, the analgesic compositions described herein may be prepared by wet mixing/granulating. Generally, the analgesic compositions may be prepared by combining (a) acetaminophen and (b) at least three of components chosen from (i) a bioavailable oral opioid; (ii) a non-steroidal anti-inflammatory drug; (iii) a sulfur-containing, N-acetyl-p-benzoquinone imine scavenging agent or prodrug thereof; or (iv) an agent which lowers acetaminophen toxicity, optionally in the presence of one or more additional components, and mixing or granulating the mixture. Techniques that may be utilized for dry mixing/granulating, including, without limitation, milling, compacting (such as roller compaction), slugging, or a combination thereof. The components may be in extragranular or intragranular phases, as determined by one of skill in the art and as determined by the requirements of the process.

When the process includes compacting and/or milling, compactors and mills known to those of skill in the art are utilized. Milling is typically performed on particles of varying sizes, i.e., large particles, powders, and fine powders, to ensure a product having preferential and uniform particle size. Milling can include several separating, recycling, and screening steps to obtain the desired particle sizes. The term “roller compaction” as used herein refers to the process of compacting two or more solid materials between rotating rolls. In one embodiment, the rolls are counter-rotating rolls and form solid ribbons of the compacted solid materials. These ribbons are then subject to further steps including milling to form an analgesic composition.

The term “slugging” as used herein refers to a process of compressing two or more solid materials on a press. In one embodiment, the press is larger than those presses utilized to prepare large tablets. These tablets are then be subject to further steps including milling to form an analgesic composition.

A variety of apparatuses and process materials may be utilized to perform the process described herein. Such materials include, without limitation, bags, screens, and blenders, among others, all of varying sizes.

The analgesic compositions desirably contain particles of an optimal size to permit dissolution of the analgesic composition, and more desirably, the particles are less than or equal to about 100 μm. The sizes of the particles of the analgesic composition are typically measured by passing the solid analgesic composition through screens of varying sizes. In one embodiment, about 8% of the particles are greater than or equal to about 350 μm. In another embodiment, about 28% of the particles are greater than or equal to about 180 μm. In a further embodiment, about 34% of the particles are greater than or equal to about 150 μm. In still another embodiment, about 39% of the particles are greater than about 125 μm. In yet another embodiment, about 49% of the particles are greater than about 89 μm. In a further embodiment, about 64% of the particles are greater than about 75 μm. In still another embodiment, about 90% of the particles are greater than about 45 μm. If the particles of the analgesic compositions are larger than the optimal size and if the same have not yet been encapsulated in a capsule, the same can be subject to further milling and screening steps, among others, to reduce the particle size.

The process may also include compressing the analgesic composition into a form suitable for oral administration as discussed below. When compressed into a form suitable for oral administration, one of skill in the art would readily be able to select a suitable press for use herein. However, one example of such a press includes the Stokes® B2 Tablet Press, among others.

Desirably, the components of the analgesic compositions described herein are purified. The term “purified” as used herein refers to a component that contains less than about 1% by wt/wt impurities. In one example, components of the analgesic compositions described herein contain less than about 0.5% wt/wt impurities. In another example, components of the analgesic compositions described herein contain less than or equal to about 0.4% impurities. In one embodiment, components of the analgesic compositions described herein are at least about 99% pure. In another embodiment, components of the analgesic compositions described herein are at least about 99.5% pure. In a further embodiment, components of the analgesic compositions described herein are at least about 99.6% pure. In yet another embodiment, components of the analgesic compositions described herein are at least about 99.7% pure. In still a further embodiment, components of the analgesic compositions described herein are at least about 99.8% pure. In another embodiment, components of the analgesic compositions described herein are at least about 99.9% pure. In still a further embodiment, components of the analgesic compositions described herein are about 100% pure.

Also envisioned by the analgesic composition are salts of any of the noted components. The term “salt” as used herein is understood to those skilled in the art and refers to salts which are pharmaceutically acceptable to a patient in need thereof. One of skill in the art would readily be able to form salts of any of the noted components using techniques and skill in the art. Such pharmaceutically acceptable salts may be formed from acids and bases.

Physiologically acceptable acids of one or more of the components of the analgesic compositions described herein include those derived from inorganic and organic acids. A number of inorganic acids are known in the art and include, without limitation, hydrochloric, hydrobromic, hydroiodic, sulfuric, nitric, phosphoric acid, hydrofluoric, boric, and perchloric. A number of organic acids are also known in the art and include, without limitation, lactic, formic, acetic, fumaric, citric, propionic, oxalic, succinic, glycolic, glucuronic, maleic, furoic, glutamic, benzoic, anthranilic, salicylic, tartaric, malonic, mallic, phenylacetic, mandelic, embonic, methanesulfonic, ethanesulfonic, panthenoic, benzenesulfonic, toluenesulfonic, stearic, sulfanilic, alginic, and galacturonic acids.

Physiologically acceptable bases include those derived from inorganic and organic bases. A number of inorganic bases are known in the art and include, without limitation, carbonates, bicarbonates, hydroxides, aluminum, calcium, lithium, magnesium, potassium, sodium, and zinc sulfate or phosphate compounds, among others. Organic bases include, without limitation, N,N,-dibenzylethylenediamine, chloroprocaine, choline, diethanolamine, ethylenediamine, meglumine, procaine, alkylamines, cyclic amines, arylamines, ammonia, alkyllithiums, amidures, among others.

Physiologically acceptable alkali salts and alkaline earth metal salts include, without limitation, sodium, potassium, calcium and magnesium salts, optionally in the form of esters, and carbamates.

The salts can be also in the form of esters, carbamates sulfate, oximes, sulfamites, carbonates and other conventional “pro-drug” forms, which, when administered in such form, convert to the active moiety in vivo. In one embodiment, the prodrugs are esters. Other forms of the components of the analgesic compositions described herein such as polymorphs or metabolites may be utilized.

The components of the analgesic compositions described herein may be milled and/or micronized. In one embodiment, the components of the analgesic compositions described herein are milled or micronized under nitrogen. Conventional milling and micronizing techniques may be utilized. In one embodiment, micronization may be performed using a Trost or jet mill, applied to the non-micronized components of the analgesic compositions described herein. Briefly, one or more of the components of the analgesic compositions described herein are micronized, desirably under nitrogen and by means of conventional micronizing techniques, for example with a Trost or jet mill.

The analgesic compositions described herein alone or in combination with an additional component and/or excipient described above are stable over 6 months. In one embodiment, the analgesic compositions described herein alone or in combination with an additional component and/or excipient described above are stable at elevated temperatures. In another embodiment, the analgesic compositions described herein or in combination with an additional component and/or excipient described above are stable over varying relative humidities (RH). In a further embodiment, the analgesic compositions described herein or in combination with an additional component and/or excipient described above are stable for 6 months at 40° C. and 75% RH. In yet another embodiment, the analgesic compositions described herein or in combination with an additional component and/or excipient described above are stable for more than 2 years at 25° C. and 65% RH.

C. Pharmaceutical Products

The analgesic compositions may be in any form suitable by oral delivery. Such forms include, without limitation, solids, liquids, and gels. In one embodiment, the analgesic composition is a solid. In another embodiment, the solid is a tablet, a caplet, or a quick dissolving film. In another embodiment, the analgesic composition is a liquid. In another embodiment, the analgesic composition is added to a capsule.

The analgesic compositions described herein may be formulated into a dosing unit for delivery to a patient. In one embodiment, the analgesic is formulated for oral delivery to a patient. Suitable dosing units include oral dosing units, such as a tablet, caplet, hard and soft gelatin capsule, gelatin free-capsule, tablet-in-capsule, caplet-in-capsule, powder, suspension, microcapsule, dispersible powder, granule, powder, liquid, suspension, syrup, elixir, bioadhesive films, chewing gum, quick dissolving film, wafer, among others. These dosing units are readily prepared using the methods described herein and those known to those of skill in the art. The dosing unit is a drug delivery device formulated for administration of the analgesic compositions described herein to a patient in need thereof. In one embodiment, the drug delivery device contains analgesic compositions described herein. The drug delivery device may be, without limitation, designed for immediate release of one or more components of the device, i.e., an immediate release unit, or is designed for controlled and delayed release of one or more components of the device, i.e., a controlled release unit.

The tablets as described herein may be utilized with or without encapsulation. In one embodiment, the tablet prepared as described herein is encapsulated in a capsule. Desirably, the capsule is a hydroxypropyl methylcellulose, hypromellose, Gelatine, or Pullulan capsule. The capsule can be optionally sealed with the tablet therein or a filler can be added to the capsule containing tablet. In one embodiment, the filler includes MCC, croscarmellose sodium, magnesium stearate, lactose, cellulose, and/or talc, among others. In one embodiment, the tablet is placed in the capsule prior to adding the filler.

Optionally, the tablets are film-coated and suitable film-coatings are known and may be prepared using techniques known to those of skill in the art. In one embodiment, the film-coating can be prepared such as suitable polymers including, without limitation, methyl cellulose, hydroxypropyl cellulose, hydroxpropylmethylcellulose, ethyl cellulose, polyvinyl alcohol, and combinations thereof. In another embodiment, the film coating is the Eudragit film. Other suitable film-coatings can be readily selected by one of skill in the art. In another embodiment, the film coating is the Opadry® seal coat. Where applied, the weight percent of the film coat is generally in the range of from 2% wt/wt to 6% wt/wt of the tablet, or integers, fractions, or ranges there between. In a further embodiment, the film coating improves the stability of analgesic composition. In another embodiment, the film coating provides an additional barrier function.

The amount of the one or more components of the analgesic compositions described herein that is effective in the methods described herein can be determined by standard clinical techniques. A pharmaceutically effective amount of one or more of the components of the analgesic compositions described herein can vary depending on the components of the analgesic composition, delivery mode, condition severity, the agent and weight of the patient, among others. The dosing regimen can also be adjusted to provide the optimal therapeutic response. A single dose can be delivered or several divided doses can be delivered daily, e.g., in divided doses 2 to 4 times a day. The dose can be reduced or increased as indicated by the exigencies of the therapeutic situation. In one embodiment, the delivery is on an hourly, daily, weekly, monthly, quarterly, or yearly basis. In another embodiment, the delivery is on a daily delivery.

Treatment can be initiated with small dosages less than the optimum dose of one or more components of the analgesic compositions described herein. The dosage may be increased until the optimum effect under the circumstances is reached. Precise dosages will be determined by the administering physician based on experience with the individual subject treated.

D. Methods of Using the Analgesic Compositions

As discussed above, the analgesic compositions herein are safe and extremely effective in treating or preventing ailments requiring treatment with acetaminophen. In doing so, the methods described herein inhibit formation, reduce levels, or reduce the toxicity of N-acetyl-p-benzoquinone imine, i.e., the toxic acetaminophen by-product, without irreparable hepatic toxicity effects. The phrase “irreparable hepatic toxicity” as used herein refers to lasting hepatic injury or liver failure as a result of acetaminophen toxicity. Desirably, the analgesic composition is administered to the patient so that excessive self-administration with multiples of the maximal recommended doses do not result in serious risk of irreparable hepatic toxicity effects.

Such methods, therefore, result in the prevention or minimization of acetaminophen liver toxicity, i.e., acetaminophen induced hepatotoxicity. In one embodiment, the analgesic composition has an at least a 150% increase in the hepatic tolerability of the combination relative to acetaminophen alone. In another embodiment, the hepatotoxic effects of acetaminophen administration are further reduced. In a still further embodiment, this reduction of hepatotoxic effects is assessed by comparing with the hepatotoxicity of an equal amount of a composition containing acetaminophen and oxycodone, but lacking one or more of a NSAID, a sulfur-containing, N-acetyl-p-benzoquinone imine scavenging agent or prodrug thereof, or an agent which lowers acetaminophen toxicity. In another embodiment, the risks of hepatotoxic effects of acetaminophen administration with self-administration of excessive doses are reduced. These risks are reduced by comparing with the hepatotoxicity of an equal amount of a composition containing acetaminophen and oxycodone, but lacking one or more of a NSAID, a sulfur-containing, N-acetyl-p-benzoquinone imine scavenging agent or prodrug thereof, or an agent which lowers acetaminophen toxicity in the proper proportions.

In one embodiment, the ailment is pain and can result from a number of conditions. The term “pain” as used herein includes all types of pain. In one embodiment, the pain may be acute or chronic and may be nociceptive, dysfunctional, idiopathic, neuropathic, somatic, visceral, inflammatory, and/or procedural. For example, the pain may be from a migraine, back pain, neck pain, gynecological pain, pre-labor or labor pain, orthopedic pain, post-stroke pain, post-surgical or procedural pain, post herpetic neuralgia, sickle cell crises, interstitial cystitis, urological pain, dental pain, headache, pain from a wound or from a medical procedure such as surgery, suturing, setting a fracture, biopsy, cancer, inflammation, nerve compression, and mechanical forces resulting from tissue distension, among others.

The methods described herein include administering an analgesic composition described herein to a patient in need thereof. A pharmaceutically effective amount of the analgesic composition is administered to a patient in need thereof. In one embodiment, the analgesic composition is self-administered by the patient. In another embodiment, the analgesic composition is administered by another individual, physician, nurse, caregiver or the like.

The methods also encompass patients administering safe doses of the prescribed analgesic composition and patients administering addictive doses of the prescribed analgesic composition, i.e., abusers. In a further embodiment, the patient administers more than one dose of the analgesic composition. In yet another embodiment, the patient administers more than the daily maximum dosage of the analgesic composition as a consequence of drug abuse or accident.

The term “abuser” as used herein refers to a patient who is prescribed an analgesic composition described herein, but administers more than the recommended dosage in one administration or in several administrations over the period of one day, i.e., an excessive dose. The abusive administration may be accidental or intentional.

The term “maximum dosage” of the analgesic composition is based on the dosage which is deemed to be safe to the average population and effectively non-toxic, i.e., a proper dosage over a specific period of time. The maximum dosage may be based on one component of the analgesic composition, more than one component of the analgesic composition, or all components of the analgesic composition. The maximum dosage relies on all components of the analgesic composition and the specifics of the condition being treated, among others. In another embodiment, the maximum dosage is about 4 to about 12 dosages, or integers, fractions, or ranges there between, containing the analgesic composition per day. In a further embodiment, the maximum dosage is about 4, 5, 6, 7, 8, 9, 10, 11, or 12 dosages per day.

As used herein, an “excessive dose” is one that exceeds the maximum dosage of any one of the components of the analgesic composition. The excessive dosage may be based on one component of the analgesic composition, more than one component of the analgesic composition, or all components of the analgesic composition. In one embodiment, the patient administers more than the maximum dosage of any one of the components of the analgesic composition. In a further embodiment, the patient administers at least 2 times of the maximum dosage of the analgesic composition. In another embodiment, an excessive dose of oxycodone or hydrocodone is greater than about 60 mg in a 24 hour period. In a further embodiment, an excessive dose of codeine is about 120 to about 320 mg, or integers, fractions, or ranges there between, in a 24 hour period. In still a further embodiment, an excessive dose of codeine is about 120, 130, 140, 150, 160, 170, 180, 190, 200, 210, 220, 230, 240, 250, 260, 270, 280, 290, 300, 310, or 320 mg in a 24 hour period. In another embodiment, an excessive dose of acetaminophen is greater than about 3,900 mg in a 24 hour period. In still a further embodiment, an excessive dose of acetaminophen is about 3,900 mg to about 8,000 mg, or integers, fractions, or ranges there between, in a 24 hour period. In another embodiment, an excessive dose of acetaminophen is about 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, or 8,000 mg. In yet another embodiment, an excessive dose of aspirin is about 10,000 mg or greater. In a further embodiment, an excessive dose includes a composition containing about 240 mg oxycodone, about 6,000 mg aspirin, about 6,000 mg acetaminophen, about 600 mg of an agent which lowers acetaminophen toxicity cimetidine, and about 1,200 mg of a sulfur-containing, N-acetyl-p-benzoquinone imine scavenging agent or prodrug thereof to a patient over a period of about 24 hours.

The terms “proper proportion” and “recommended dose”, or variations thereof as used herein, are used interchangeably. In one embodiment, a proper proportion of the opioid is about 5 to about 10 mg, or integers, fractions, or ranges there between, per dose. In a further embodiment, a proper proportion of the opioid is about 5, 6, 7, 8, 9, or 10 mg per dose. In another embodiment, a proper proportion of the opioid is about 5 mg per dose. In a further embodiment, a proper proportion of the opioid is about 20 to about 60 mg, or integers, fractions, or ranges there between, in a 24 hour period. In another embodiment, a proper proportion of the opioid is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg in a 24 hour period. In yet another embodiment, a proper proportion of acetaminophen is about 250 to about 650 mg, or integers, fractions, or ranges there between, per dose. In a further embodiment, a proper proportion of acetaminophen is about 250, 300, 350, 400, 450, 500, 550, 600, or 650 mg per dose. In still a further embodiment, a proper proportion of acetaminophen is about 1,000 to about 3,900 mg, or integers, fractions, or ranges there between, in a 24 hour period. In yet another embodiment, a proper proportion of acetaminophen is about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, or 3,900 mg in a 24 hour period.

The methods described herein are effective in reducing acetaminophen induced hepatotoxicity for even one dose of an analgesic composition which contains acetaminophen. The methods and compositions discussed herein are effective in the absence or presence of alcohol. In one embodiment, the analgesic composition is administered in more than one dose. In another embodiment, the analgesic composition is administered to the patient over an extended period of time. The phrase “extended period of time” as used herein refers to a period of time that exceeds a typical period of usage. In one embodiment, the extended period of time includes a period that exceeds one week, a period that exceeds one month, or a period that exceeds one year.

The methods thereby protect patients, including abusers, from ingesting a wide range amount of the analgesic composition without risking hepatotoxic injury. In one embodiment, the method includes administering 1 to about 12 tablets, or integers, fractions, or ranges there between, containing the analgesic composition to a patient over a period of about 24 hours. In another embodiment, the method includes administering about 120 mg of the opioid, about 3,000 mg of the NSAID, 3,000 mg of acetaminophen, 300 mg of an agent which lowers acetaminophen toxicity, and 600 mg of a sulfur-containing, N-acetyl-p-benzoquinone imine scavenging agent or prodrug thereof to a patient over a period of about 24 hours. In a further embodiment, the method includes administering about 240 mg of the opioid, 6,000 mg of the NSAID, 6,000 mg of acetaminophen, 600 mg of a sulfur-containing, N-acetyl-p-benzoquinone imine scavenging agent or prodrug thereof the methods are non-toxic.

In one embodiment, a method for inhibiting formation or reducing levels of N-acetyl-p-benzoquinone imine and includes administering an analgesic composition described herein to a patient in need thereof.

In another embodiment, a method for inhibiting formation or reducing toxicity of N-acetyl-p-benzoquinone imine is provided and includes administering an analgesic composition described herein to a patient in need thereof.

In a further embodiment, a method for preventing or minimizing acetaminophen liver toxicity is provided and includes administering an analgesic composition described herein to a patient in need thereof.

In yet another embodiment, a method for preventing or treating pain is provided and includes administering an analgesic composition described herein to a patient in need thereof. In one embodiment, the pain is acute pain, chronic pain (such as, for example, neuropathic pain), or post-surgical pain. In still a further embodiment, a method for treating pain or an ailment in a patient requiring treatment with acetaminophen is provided and includes administering at least one dose of an analgesic composition described herein to the patient. In this method, the hepatotoxic effects of acetaminophen administration are reduced with respect to administration of an equal amount of a composition containing acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity. Advantageously, the analgesic composition can be administered to the patient without irreparable hepatic toxicity effects.

In another embodiment, a method for treating pain or an aliment in a patient requiring treatment with acetaminophen is provided and includes administering an analgesic composition described herein to the patient. In this method, the risks of hepatotoxic effects of acetaminophen administration with self-administration of excessive doses are reduced with respect to administration of an equal amount of a composition containing acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity in the proper proportions.

Advantageously, the analgesic composition can be administered to the patient so that excessive self-administration with multiples of the maximal recommended doses do not result in serious risk of irreparable hepatic toxicity effects. In one aspect, the proper proportion of the opioid is about 5 to about 10 mg per dose, or integers, fractions, or ranges there between. In another aspect, the proper proportion of the opioid is about 5 mg per dose. In a further aspect, the proper proportion of the opioid is from about 20 to about 60 mg, or integers, fractions, or ranges there between, in a 24 hour period. In still another aspect, an excessive dose of oxycodone or hydrocodone is greater than about 60 mg in a 24 hour period. In yet a further aspect, an excessive dose of codeine is from about 120 to about 320 mg, or integers, fractions, or ranges there between, in a 24 hour period. In another aspect, the proper proportion of acetaminophen is from about 250 to about 650 mg, or integers, fractions, or ranges there between, per dose. In a further aspect, the proper proportion of acetaminophen is from about 1,000 to about 3,900 mg, or integers, fractions, or ranges there between, in a 24 hour period. In still another aspect, an excessive dose of acetaminophen is greater than about 3,900 mg in a 24 hour period. In yet another aspect, an excessive dose of acetaminophen is from about 3,900 mg to about 8,000 mg, or integers, fractions, or ranges there between, in a 24 hour period.

In yet a further embodiment, a method for decreasing acetaminophen induced hepatotoxicity is provided and includes administering an analgesic composition described herein to a patient in need therein. In one aspect, acetaminophen is administered to the patient over an extended period of time. In another embodiment, the extended period of time is a period that exceeds one week, a period that exceeds one month, or a period that exceeds one year.

E. Kits

Also provided are kits or packages containing the analgesic compositions described herein. In one embodiment, the kit contains a dosing unit containing analgesic compositions described herein. A number of packages or kits for use in dispensing pharmaceutical agents for are known in the art. In one embodiment, the kit is formulated for oral administration. In a further embodiment, the kit contains one or more dosing forms containing the analgesic composition suitable for oral administration. In another embodiment, the kits are identical, i.e., contain the same number of drug delivery agents and dosages thereof. In another embodiment, the kits contain different numbers of drug delivery agents and/or dosages thereof

In another embodiment, the kit has indicators for each day of administration. When the analgesic compositions described herein are delivered with periodic discontinuation, a package or kit can include a pharmaceutically acceptable placebo on those days when the analgesic composition described herein is not delivered. The term “placebo” as used herein refers to a pharmaceutically acceptable chemical compound or composition which is pharmaceutically inactive in the patient for the indicated treatment. One of skill in the art would readily be able to select a suitable placebo for use herein.

“Controlled release”, as used herein, refers to timed release of any one of the components of the analgesic compositions described herein. In one embodiment, release of any one or more of the components of the analgesic compositions described herein is over a period of at least 1 hour. In another embodiment, release of the any one or more of the components of the analgesic compositions described herein is over a period of at least 2 hours. In a further embodiment, the release of the any one or more of the components of the analgesic compositions described herein is over a period of at least 3 hours. In yet another embodiment, release of the any one or more of the components of the analgesic compositions described herein is over a period of at least 4-6 hours. When formulated as a controlled release unit, the drug delivery device may be a tablet, caplet, capsule, tablet-in-capsule, powder, liquid, thin film wafer, lollipop, or bioadhesive buccal.

The kit contains at least one dosage unit for each component of the kit. In one embodiment, the kit contains at least one dosage unit of the analgesic compositions described herein. In a further embodiment, the kit contains one dosage unit of a pharmaceutically acceptable placebo as described above. If the concentration of one or more component of the kit is varied, the kit may contain a sequence of the dosage units.

In one embodiment, the daily dosage of one or more components of the analgesic compositions described herein remains fixed in each phase. In another embodiment, the daily dosage of one or more of the components of the analgesic compositions described herein varies in each phase. The daily dose units described may be delivered in the order described, with the first phase followed in order by the second and third phases, etc., or may be varied.

The kit can further contain instructions for administering the analgesic compositions described herein alone or in combination with one or more instruments including, without limitation, syringe, pipette, forceps, measuring spoon, or the like. Other components for inclusion in the kits would be clear to those skilled in the art, taking into consideration the desired indication and mode of delivery.

The kit desirably contains, in all of the containers included therein, drug delivery devices totaling the number of days for their administration.

In one embodiment, a kit is provided and contains (A) an oral dosage unit comprising an analgesic composition described herein and (B) instructions for administering the oral dosage unit. In one aspect, the instructions include administering one to two of the oral dosage units every about 4 to about 6 hours, or integers, fractions, or ranges there between. In another aspect, the instructions include administering no more than 12 tablets in 24 hours.

In another embodiment, a kit is provided and includes an oral dosage unit which contains (a) about 250 mg of acetaminophen; (b)(i) about 10 mg of oxycodone, hydrocodone, or codeine; (b)(ii) about 250 mg of aspirin; (b)(iii) about 50 mg of cimetidine or disulfiram; and (b)(iv) about 100 mg of methionine.

In another embodiment, a kit is provided and includes an oral dosage unit which (a) contains about 250 mg of acetaminophen; (b)(i) about 10 mg of oxycodone, hydrocodone, or codeine; (b)(ii) about 250 mg of aspirin; (b)(iii) about 25 mg of cimetidine or disulfiram; and (b)(iv) about 50 mg of methionine.

In a further embodiment, a kit is provided and includes an oral dosage unit which contains (a) about 250 mg of acetaminophen; (b)(i) about 30 mg of codeine; (b)(ii) about 250 mg of aspirin; (b)(iii) about 25 mg of cimetidine or disulfiram; and (b)(iv) about 50 mg of methionine.

In yet another embodiment, a kit is provided and includes an oral dosage unit which contains (a) about 250 mg of acetaminophen; (b)(i) about 10 mg of oxycodone, hydrocodone, or codeine; (b)(ii) about 200 mg of ibuprofen; (b)(iii) about 25 mg of cimetidine or disulfiram; and (b)(iv) about 50 mg of methionine.

The following examples are illustrative only and are not intended to limit the present invention.

### EXAMPLES

### Example 1—Analgesic Compositions

Analgesic compositions are prepared according to Table A by combining the noted components Pharmaceutically acceptable excipients are also included in the analgesic compositions and include, without limitation, one or more of benzoic acid, carnauba wax, castor oil, colloidal silicon dioxide, croscarmellose sodium, crospovidone hydroxyethyl cellulose, hydroxypropylcellulose, hydroxypropyl methylcellulose, hypromellose, lactose, magnesium stearate, microcrystalline cellulose, mineral oil, modified starch, polyethylene glycol, polysorbate 20, povidone, powdered cellulose, pregelatinized starch, propylene glycol, shellac, simethicone emulsion, sodium metabisulfite sodium starch glycolate, sorbitan monolaurate, stearic acid, sucralose, titanium dioxide, or triacetin.

TABLE A

| | Acetamin- | | Hydro- | Methi- | Cimet- |
|---|---|---|---|---|---|
| Compo- | ophen | Aspirin | codone | onine | idine |
| sition | (mg) | (mg) | (mg) | (mg) | (mg) |
| 1 | 250 | 250 | 5 | 50 | 25 |
| 2 | 250 | 250 | 7.5 | 75 | 35 |
| 3 | 250 | 250 | 10 | 100 | 50 |
| 4 | 250 | 250 | 5 | 50 | 0 |
| 5 | 250 | 250 | 7.5 | 75 | 0 |
| 6 | 250 | 250 | 10 | 100 | 0 |
| 7 | 250 | 250 | 5 | 0 | 25 |
| 8 | 250 | 250 | 7.5 | 0 | 35 |
| 9 | 250 | 250 | 10 | 0 | 50 |
| 10 | 300 | 300 | 5 | 50 | 25 |
| 11 | 300 | 300 | 7.5 | 75 | 35 |
| 12 | 300 | 300 | 10 | 100 | 50 |
| 13 | 300 | 300 | 5 | 50 | 0 |
| 14 | 300 | 300 | 7.5 | 75 | 0 |
| 15 | 300 | 300 | 10 | 100 | 0 |
| 16 | 300 | 300 | 5 | 0 | 25 |
| 17 | 300 | 300 | 7.5 | 0 | 35 |
| 18 | 300 | 300 | 10 | 0 | 50 |
| 19 | 325 | 325 | 5 | 50 | 25 |
| 20 | 325 | 325 | 7.5 | 75 | 35 |
| 21 | 325 | 325 | 10 | 100 | 50 |
| 22 | 325 | 325 | 5 | 50 | 0 |
| 23 | 325 | 325 | 7.5 | 75 | 0 |
| 24 | 325 | 325 | 10 | 100 | 0 |
| 25 | 325 | 325 | 5 | 0 | 25 |
| 26 | 325 | 325 | 7.5 | 0 | 35 |
| 27 | 325 | 325 | 10 | 0 | 50 |
| 28 | 400 | 400 | 5 | 50 | 25 |
| 29 | 400 | 400 | 7.5 | 75 | 35 |
| 30 | 400 | 400 | 10 | 100 | 50 |
| 31 | 400 | 400 | 5 | 50 | 0 |
| 32 | 400 | 400 | 7.5 | 75 | 0 |
| 33 | 400 | 400 | 10 | 100 | 0 |
| 34 | 400 | 400 | 5 | 0 | 25 |
| 35 | 400 | 400 | 7.5 | 0 | 35 |
| 36 | 400 | 400 | 10 | 0 | 50 |
| 37 | 500 | 500 | 5 | 50 | 25 |
| 38 | 500 | 500 | 7.5 | 75 | 35 |
| 39 | 500 | 500 | 10 | 100 | 50 |
| 40 | 500 | 500 | 5 | 50 | 0 |
| 41 | 500 | 500 | 7.5 | 75 | 0 |
| 42 | 500 | 500 | 10 | 100 | 0 |
| 43 | 500 | 500 | 5 | 0 | 25 |
| 44 | 500 | 500 | 7.5 | 0 | 35 |
| 45 | 500 | 500 | 10 | 0 | 50 |
| 46 | 650 | 650 | 5 | 50 | 25 |
| 47 | 650 | 650 | 7.5 | 75 | 35 |
| 48 | 650 | 650 | 10 | 100 | 50 |
| 49 | 650 | 650 | 5 | 50 | 0 |
| 50 | 650 | 650 | 7.5 | 75 | 0 |
| 51 | 650 | 650 | 10 | 100 | 0 |
| 52 | 650 | 650 | 5 | 0 | 25 |
| 53 | 650 | 650 | 7.5 | 0 | 35 |
| 54 | 650 | 650 | 10 | 0 | 50 |

These analgesic compositions are then formulated into an oral dosing unit such as a tablet, caplet, or capsule for administration to a patient in need thereof.

The stability of these analgesic compositions, prior to and after formation of the oral dosing unity, is then measured using standard techniques known in the art. It is expected that these analgesic compositions, and oral dosing units thereof, will be stable over a period of about 1 month for samples stored at varying temperatures and humidities according to Food and Drug Administration standards.

### Example 2—Toxicity in Mice

This example is performed as described in Saito, Hepatology, 51(1):246, 2010, which is incorporated by reference herein. Fasted male C3HeB/FeJ mice (8-10 weeks old) or equivalent receive 200-400 mg/kg acetaminophen intraperitoneally in a dose ranging pilot phase to determine the exact conditions to demonstrate acetaminophen toxicity in a single dose trial using plasma ALT activity as the outcome measure.

At selected times after acetaminophen treatment, groups of animals are killed by way of cervical dislocation under anesthesia. Blood is drawn from the vena cava into heparinized syringes and centrifuged. The plasma is used for determination of alanine aminotransferase (ALT) activities. Plasma ALT activities are determined and expressed as international units per liter.

After establishing the model in the pilot phase, five experimental groups are studied. The groups receive vehicles containing the ingredients noted in Table B. If the methionine or cimetidine is ineffective or is fully protective, the doses are adjusted and the experiment repeated until the relative contribution of each relative to the combination is determined.

TABLE B

Components

| | Acetamin- | Cimet- | Methi- |
|---|---|---|---|
| Group | ophen | idine | onine |
| A | yes | No | No |
| B | yes | 20% of the | No |
| | | acetamin- | |
| | | ophen dose | |
| C | yes | No | 30% of the |
| | | | acetamin- |
| | | | ophen dose |
| D | yes | 20% of the | 30% of the |
| | | acetamin- | acetamin- |
| | | ophen dose | ophen dose |

The outcome is maximal ALT elevation. Data are expressed as the mean±standard error. Comparison between groups is performed analysis of variance, or alternative non-parametric procedures if the data is not normally distributed. P<0.05 is considered significant.

It is expected that the data will illustrate that the acetaminophen alone causes liver injury. While the liver is protected against damage when the treatments contain cimetidine or methionine alone, it is expected that higher levels of protection will be observed for compositions containing both cimetidine and methionine.

### Example 3—Toxicity in Rats

This example is performed as described in Speeg, Journal Pharmacol. Exp. Ther., 234:550-554, 1985, which is incorporated by reference herein. Male Wistar rats or equivalent are pre-treated with 3-methylcholanthrine (3MC) to induce hepatic oxidation of xenobiotics. They are then given a single dose of 300-600 mg/kg acetaminophen IP in a pilot phase to establish optimal experimental conditions. Twenty-four hours later, samples are taken for serum transaminase determinations.

Once optimal assay conditions are established, five groups of animals are administered the compositions set forth in Table C. If the methionine or the cimetidine is ineffective or is fully protective, the doses are adjusted and the experiment repeated until the relative contribution of each relative to the combination is determined.

TABLE C

Components

| | | Acetamin- | Cimet- | Methi- |
|---|---|---|---|---|
| Group | Vehicle | ophen | idine | onine |
| A | yes | No | No | No |
| B | yes | yes | No | No |
| C | yes | yes | 20% of the | No |
| | | | acetamin- | |
| | | | ophen dose | |
| D | yes | yes | No | 30% of the |
| | | | | acetamin- |
| | | | | ophen dose |
| E | yes | yes | 20% of the | 30% of the |
| | | | acetamin- | acetamin- |
| | | | ophen dose | ophen dose |

The outcome is maximal ALT elevation. Data are expressed as the mean±standard error. Comparison between groups is performed analysis of variance, or alternative non-parametric procedures if the data is not normally distributed. P<0.05 is considered significant.

It is expected that the test mixtures result in reduced acute toxicity of the acetaminophen as measured by transaminase levels, and that the effect of both treatments combined prove superior to each alone.

### Example 4—Toxicity in Humans

Thirty-six volunteers (age=18-45 years, weight=50-100 kg) participate in a six week, single-blind, two-period study of the relative laboratory toxicity of oral acetaminophen versus a combination of acetaminophen, cimetidine and methionine.

All subjects participate in an initial multiple dose study of 1 gram of acetaminophen given four times a day (four grams a day), protected by twice a day transaminase determinations. All subjects developing a transaminase elevation 3 times the upper limit of normal or greater immediately stop acetaminophen administration, receive oral methionine rescue, and are eligible for the second phase of the study. Subjects with no elevation are terminated from the study. Subjects rest for three weeks after the first phase and then return for the second phase.

After returning with normal transaminase values, and after an overnight fast, subjects are randomized to receive either (i) capsules containing 500 mg of acetaminophen and dextrose filler or (ii) capsules containing 500 mg of acetaminophen, 50 mg of cimetidine and 150 mg methionine. Subjects dose at 4 grams of acetaminophen a day. They have twice daily blood samples for serum transaminases, and proceed to the next day's dosing only if their most recent transaminase values are no more than 3 times the upper limit of normal. Subjects with transaminase values more than three times the upper limit of normal (ULN) stop acetaminophen dosing immediately, receive 1000 mg of oral methionine four times a day for 24 hours and have daily transaminase determinations until the values return to normal.

The study conditions provide minimum risk to the subjects, such that the statistical signal for this study is small. The outcome variable is maximal transaminase elevations for each subject relative to baseline tested by ANOVA for each formulation. The test article is hepato-protective if there is a significant (P<0.05) difference in outcome for the formulation.

Subjects with transaminase elevations are followed at intervals to ensure that their values return to normal.

It is expected that the subjects complete the study safely, no additional serious adverse effects result from the methionine and cimetidine and that the antidotal compositions result in reduced acute toxicity of the acetaminophen in the subjects.

### Example 5—Pharmacokinetic Studies

(i) Study 1

The first study compares the bioequivalence of oxycodone when formulated in a composition containing only acetaminophen, against a formulation containing aspirin, cimetidine and methionine as discussed herein. This is a conventional single-dose, cross-over, pharmacokinetic study in twelve normal volunteers comparing Cₘₐₓ, Tₘₐₓ and AUC of oxycodone and is performed as described in 21 CFR §320, “Bioavailability and Bioequivalence Requirements”, 2014, which is incorporated by reference herein. The test articles are commercially available capsules which contain the components set forth in Table D.

TABLE D

| | Components | | | | |
|---|---|---|---|---|---|
| | Oxyco- | Acetamin- | | Methi- | Cimet- |
| Test | done | ophen | Aspirin | onine | idine |
| Article | (mg) | (mg) | (mg) | (mg) | (mg) |
| 1 | 5 | 325 | 0 | 0 | 0 |
| 2 | 5 | 325 | 325 | 130 | 65 |

It is expected that test articles 1 and 2 will be bio-equivalent.

(ii) Study 2

This second study compares the pharmacokinetics of aspirin and acetaminophen in a marketed analgesic combination containing acetaminophen and aspirin relative to an aspirin and acetaminophen combination containing acetaminophen, aspirin, cimetidine and methionine. This is a conventional single-dose, cross-over, pharmacokinetic study in twelve normal volunteers comparing Cₘₐₓ, Tₘₐₓ and AUC of the aspirin and the acetaminophen and is performed as described in 21 CFR §320, “Bioavailability and Bioequivalence Requirements”, 2014, which is incorporated by reference herein. The test articles are commercially available capsules which contain the components set forth in Table E.

TABLE E

Components

| | Acetamin- | | Methi- | Cimet- |
|---|---|---|---|---|
| | ophen | Aspirin | onine | idine |
| Test Article | (mg) | (mg) | (mg) | (mg) |
| 1 | 250 | 250 | 0 | 0 |
| 2 | 250 | 250 | 100 | 50 |

It is expected that test articles 1 and 2 will be bio-equivalent.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

What is claimed is:

1. An analgesic composition, comprising:
(a) acetaminophen and
(b) at least three components chosen from:
(i) a bioavailable oral opioid;
(ii) a non-steroidal anti-inflammatory drug;
(iii) a sulfur-containing, N-acetyl-p-benzoquinone imine scavenging agent or prodrug thereof; or
(iv) an agent which lowers acetaminophen toxicity.

2. The analgesic composition according to claim 1, comprising (a) acetaminophen and
(b)(i) a bioavailable oral opioid;
(b)(ii) a non-steroidal anti-inflammatory drug;
(b)(iii) a sulfur-containing, N-acetyl-p-benzoquinone imine scavenging agent or prodrug thereof; and
(b)(iv) an agent which lowers acetaminophen toxicity.

3. The analgesic composition according to claim 1 or claim 2, wherein said opioid is oxycodone, hydrocodone or codeine.

4. The analgesic composition according to any one of claims 1 to 3, comprising from about 0.5 to about 16% wt/wt of said opioid.

5. The analgesic composition according to any one of claims 1 to 4, comprising from about 2.5 to about 60 mg of said opioid.

6. The analgesic composition according to any one of claims 1 to 5, comprising from about 1 to about 3% wt/wt of oxycodone or hydrocodone.

7. The analgesic composition according to any one of claims 1 to 6, comprising from about 10 mg of oxycodone or hydrocodone.

8. The analgesic composition according to any one of claims 1 to 7, wherein said non-steroidal anti-inflammatory drug is aspirin, naproxen or ibuprofen.

9. The analgesic composition according to any one of claims 1 to 8, comprising from about 10 to about 65% wt/wt of said non-steroidal anti-inflammatory drug.

10. The analgesic composition according to any one of claims 1 to 9, comprising from about 50 to about 500 mg of said non-steroidal anti-inflammatory drug.

11. The analgesic composition according to any one of claims 1 to 10, comprising from about 30 to about 45% wt/wt of aspirin.

12. The analgesic composition according to any one of claims 1 to 9, comprising about 250 mg of aspirin.

13. The analgesic composition according to any one of claims 1 to 9, comprising from about 15 to about 30% wt/wt of naproxen.

14. The analgesic composition according to any one of claim 1 to 9 or 13, comprising about 110 mg of naproxen.

15. The analgesic composition according to any one of claims 1 to 9, comprising from about 30 to about 40% wt/wt of ibuprofen.

16. The analgesic composition according to any one of claim 1 to 9 or 15, comprising about 200 mg of ibuprofen.

17. The analgesic composition according to any one of claims 1 to 16, comprising from about 20 to about 80% wt/wt of said acetaminophen.

18. The analgesic composition according to any one of claims 1 to 17, comprising from about 35 to about 65% wt/wt of said acetaminophen.

19. The analgesic composition according to any one of claims 1 to 18, comprising from about 150 mg to about 500 mg of said acetaminophen.

20. The analgesic composition according to any one of claims 1 to 19, comprising about 250 mg of acetaminophen.

21. The analgesic composition according to any one of claims 1 to 20, wherein the amount of acetaminophen is from about 20% to about 25% less than the FDA recommended maximum amount.

22. The analgesic composition according to any one of claims 1 to 21, further comprising one or more additional analgesic.

23. The analgesic composition according to any one of claims 1 to 22, wherein said agent that lowers acetaminophen toxicity is cimetidine or disulfiram.

24. The analgesic composition according to any one of claims 1 to 23, comprising from about 2 to about 20% wt/wt of cimetidine.

25. The analgesic composition according to any one of claims 1 to 24, comprising from about 2 to about 8% wt/wt of cimetidine.

26. The analgesic composition according to any one of claims 1 to 25, comprising from about 20 to about 75 mg of cimetidine.

27. The analgesic composition according to any one of claims 1 to 26, comprising about 50 mg of cimetidine.

28. The analgesic composition according to any one of claims 1 to 23, comprising from about 2 to about 8% wt/wt of disulfiram.

29. The analgesic composition according to any one of claim 1 to 23 or 28, comprising about 50 mg of disulfiram.

30. The analgesic composition according to any one of claims 1 to 29, wherein said sulfur-containing agent is methionine, glutathione, n-acetyl-cysteine, or L-cysteine.

31. The analgesic composition according to any one of claims 1 to 30, comprising from about 3 to about 35% wt/wt of said sulfur-containing agent.

32. The analgesic composition according to any one of claims 1 to 31, comprising from about 25 to about 125 mg of said sulfur-containing agent.

33. The analgesic composition according to any one of claims 1 to 32, comprising from about 6 to about 15% wt/wt of methionine.

34. The analgesic composition according to any one of claims 1 to 33, comprising about 100 mg of methionine.

35. The analgesic composition according to any one of claims 1 to 32, comprising from about 6 to about 30% wt/wt of glutathione.

36. The analgesic composition according to any one of claim 1 to 32 or 35, comprising from about 50 to about 150 mg of glutathione.

37. The analgesic composition according to any one of claims 1 to 32, comprising from about 6 to about 30% wt/wt of n-acetyl-cysteine.

38. The analgesic composition according to any one of claim 1 to 32 or 37, comprising from about 50 to about 150 mg of n-acetyl-cysteine.

39. The analgesic composition according to any one of claims 1 to 32, comprising from about 6 to about 30% wt/wt of L-cysteine.

40. The analgesic composition according to any one of claim 1 to 32 or 39, comprising from about 50 to about 150 mg of L-cysteine.

41. The analgesic composition according to any one of claims 1 to 40, wherein the amount of opioid is high related to the amount of said acetaminophen.

42. The composition according to any one of claims 1 to 41, wherein weight ratio of hydrocodone or oxycodone to acetaminophen is about 5:325 or greater.

43. The composition according to any one of claims 1 to 41, wherein the weight ratio of codeine to acetaminophen is about 30:325 or greater.

44. The analgesic composition according to claim 1 or claim 2, comprising (b)(i) oxycodone, hydrocodone, or codeine, (b)(ii) ibuprofen or aspirin, (b)(iii) cimetidine or disulfiram, and (b)(iv) methionine.

45. The analgesic composition according to claim 44, comprising:
(a) about 250 mg of acetaminophen;
(b)(i) about 10 mg of oxycodone or hydrocodone;
(b)(ii) about 250 mg of aspirin;
(b)(iii) about 25 mg of cimetidine or disulfiram; and
(b)(iv) about 50 mg of methionine.

46. The analgesic composition according to claim 44, comprising:
(a) about 250 mg of acetaminophen;
(b)(i) about 30 mg of codeine;
(b)(ii) about 250 mg of aspirin;
(b)(iii) about 25 mg of cimetidine or disulfiram; and
(b)(iv) about 50 mg of methionine.

47. The analgesic composition according to claim 44, comprising:
(a) about 250 mg of acetaminophen;
(b)(i) about 10 mg of oxycodone, hydrocodone, or codeine;
(b)(ii) about 200 mg of ibuprofen;
(b)(iii) about 25 mg of cimetidine or disulfiram; and
(b)(iv) about 50 mg of methionine.

48. The analgesic composition according to any one of claims 1 to 47, further comprising an antihistamine, a stimulant, an H1 blocker, an H2 blocker, an anti-inflammatory, a proton-pump inhibitor, a laxative, a 5-HT3-blocker, an anxiolytic, a glutathione production promoter, an antiemetic, an odor reducer, or a muscle relaxant.

49. The analgesic composition according to claim 48, wherein said glutathione production promoter is N-acetyl-cysteine.

50. The analgesic composition according to any one of claims 1 to 49, further comprising one or more of a pharmaceutically acceptable excipient.

51. The analgesic composition according to any one of claims 1 to 50, further comprising a fragrance, a coloring agent or a stability enhancing agent.

52. The analgesic composition according to claim 51, wherein said stability enhancing agent is an anti-oxidant.

53. The analgesic composition according to claim 52, wherein said anti-oxidant is butylated hydroxyl toluene, vitamin E, or a combination thereof.

54. The analgesic composition according to any one of claims claims 1 to 53, which lacks a CYP450-inducing or CYP450-stimulating additive.

55. The analgesic composition according to claim 54, wherein said CYP450-inducing or CYP450-stimulating additive comprises caffeine, theophylline, or theobromine.

56. The analgesic composition according to any one of claims 1 to 55, which is a solid.

57. The analgesic composition according to claim 56, wherein said solid is a tablet, a caplet or a quick dissolving film.

58. The analgesic composition according to any one of claims 1 to 57, which is added to a capsule.

59. The analgesic composition according to any one of claims 1 to 55, which is a liquid.

60. A method for inhibiting formation or reducing levels of N-acetyl-p-benzoquinone imine, said method comprising administering an analgesic composition of any one of claims 1 to 59 to a patient in need thereof.

61. A method for inhibiting formation or reducing toxicity of N-acetyl-p-benzoquinone imine, said method comprising administering an analgesic composition of any one of claims 1 to 59 to a patient in need thereof.

62. A method for preventing or minimizing acetaminophen liver toxicity, said method comprising administering an analgesic composition of any one of claims 1 to 59 to a patient in need thereof.

63. A method for preventing or treating pain, said method comprising administering an analgesic composition of any one of claims 1 to 59 in a patient in need thereof.

64. A method for treating pain or an ailment in a patient requiring treatment with acetaminophen, said method comprising administering at least one dose of an analgesic composition of any one of claims 1 to 59;
wherein hepatotoxic effects of acetaminophen administration are reduced with respect to administration of an equal amount of a composition comprising acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity;
wherein said analgesic composition can be administered to said patient without irreparable hepatic toxicity effects.

65. The method according to claim 64, wherein said analgesic composition is self-administered by the patient.

66. The method according to claim 64 or 65, wherein said patient administers more than one dose of said analgesic composition.

67. The method according to any one of claims 64 to 66, wherein said patient administers more than the maximum dosage of said analgesic composition.

68. The method according to claim 67, wherein said maximum dosage is about 4 to about 12 tablets per day.

69. The method according to any one of claims 64 to 68, wherein said patient administers at least 2 times of the maximum dosage of said analgesic composition.

70. The method according to any one of claims 64 to 69, wherein said irreparable hepatic toxicity effects comprises lasting hepatic injury or liver failure.

71. The method according to any one of claims 64 to 70, wherein said analgesic composition has an at least 150% increase in the hepatic tolerability of acetaminophen.

72. A method for treating pain or an ailment in a patient requiring treatment with acetaminophen, said method comprising an analgesic composition of any one of claims 1 to 59;
wherein risks of hepatotoxic effects of acetaminophen administration with self-administration of excessive doses are reduced with respect to administration of an equal amount of a composition comprising acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity in the proper proportions;
wherein said analgesic composition can be administered to said patient so that excessive self-administration with multiples of the maximal recommended doses do not result in serious risk of irreparable hepatic toxicity effects.

73. The method according to claim 72, wherein said proper proportion of said opioid is from about 5 to about 10 mg per dose.

74. The method according to claim 72 or 73, wherein said proper proportion of said opioid is about 5 mg per dose.

75. The method according to claim 72, wherein said proper proportion of said opioid is from about 20 to about 60 mg in a 24 hour period.

76. The method according to claim 72, wherein said excessive dose of oxycodone or hydrocodone is greater than about 60 mg in a 24 hour period.

77. The method according to claim 72, wherein said excessive dose of codeine is about 120 to about 320 mg in a 24 hour period.

78. The method according to any one of claims 72 to 77, wherein said proper proportion of said acetaminophen is from about 250 to about 650 mg per dose.

79. The method according to any one of claims 72 to 77, wherein said proper proportion of said acetaminophen is from about 1,000 to about 3,900 mg in a 24 hour period.

80. The method according to any one of claims 72 to 77, wherein said excessive dose of said acetaminophen is greater than about 3,900 mg in a 24 hour period.

81. The method according to any one of claims 72 to 80, wherein said excessive dose of said acetaminophen is from about 3,900 mg to about 8,000 mg in a 24 hour period.

82. A method for decreasing acetaminophen induced hepatotoxicity in a patient, said method comprising administering an analgesic composition of any one of claims 1 to 59.

83. The method according to claim 82, wherein acetaminophen is administered to said patient over an extended period of time.

84. The method according to claim 83, wherein said extended period of time is a period that exceeds one week, a period that exceeds one month, or a period that exceeds one year.

85. A kit comprising:
(A) an oral dosage unit comprising an analgesic composition of any one of claims 1 to 59; and
(B) instructions for administering said oral dosage unit.

86. The kit according to claim 85, wherein said instructions comprise administering one to two of said oral dosage units every about 4 to about 6 hours.

87. The kit according to claim 85, wherein said instructions comprise administering no more than 12 tablets in 24 hours.

88. The kit according to any one of claims 85 to 87, wherein said oral dosage unit comprises:
(a) about 250 mg of acetaminophen;
(b)(i) about 10 mg of oxycodone, hydrocodone, or codeine;
(b)(ii) about 250 mg of aspirin;
(b)(iii) about 25 mg of cimetidine or disulfiram; and
(b)(iv) about 50 mg of methionine.

89. The kit according to any one of claims 85 to 87, wherein said oral dosage unit comprises:
(a) about 250 mg of acetaminophen;
(b)(i) about 30 mg of codeine;
(b)(ii) about 250 mg of aspirin;
(b)(iii) about 25 mg of cimetidine or disulfiram; and
(b)(iv) about 50 mg of methionine.

90. The kit according to any one of claims 85 to 87, wherein said oral dosage unit comprises:
(a) about 250 mg of acetaminophen;
(b)(i) about 10 mg of oxycodone, hydrocodone, or codeine;
(b)(ii) about 200 mg of ibuprofen;
(b)(iii) about 25 mg of cimetidine or disulfiram; and
(b)(iv) about 50 mg of methionine.

91. An analgesic composition according to any one of claims 1 to 59, for use in a method for inhibiting formation or reducing levels of N-acetyl-p-benzoquinone imine in a patient.

92. An analgesic composition according to any one of claims 1 to 59, for use in a method for inhibiting formation or reducing toxicity of N-acetyl-p-benzoquinone imine in a patient.

93. An analgesic composition according to any one of claims 1 to 59, for use in a method for preventing or minimizing acetaminophen liver toxicity in a patient.

94. An analgesic composition according to any one of claims 1 to 59, for use in a method for preventing or treating pain in a patient.

95. An analgesic composition according to any one of claims 1 to 59, for use in a method for treating pain or an ailment in a patient requiring treatment with acetaminophen, wherein hepatotoxic effects of acetaminophen are reduced with respect to treatment with an equal amount of a composition comprising acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity.

96. An analgesic composition according to any one of claims 1 to 59, for use in a method for treating pain or an ailment in a patient requiring treatment with acetaminophen, wherein risks of hepatotoxic effects of acetaminophen administration with self-administration of excessive doses are reduced with respect to administration of an equal amount of a composition comprising acetaminophen and oxycodone, but lacking one or more of a non-steroidal anti-inflammatory drug, a sulfur-containing, N-acetyl-p-benzoquinone imine scavenging agent or prodrug thereof, or an agent which lowers acetaminophen toxicity in the proper proportions;
wherein excessive self-administration with multiples of the maximal recommended doses do not result in serious risk of irreparable hepatic toxicity effects.

97. An analgesic composition according to any one of claims 1 to 59, for use in a method for decreasing acetaminophen induced hepatotoxicity in a patient.
